(19) United States
(12) Patent Application Publication
(10) Pub. No.: US 2014/0088654 A1
(43) Pub. Date: Mar. 27, 2014

(54) Bone Splint

(71) Applicant: Silvana Marasco, Prahran (AU)

(72) Inventor: Silvana Marasco, Prahran (AU)

(73) Assignee: ALFRED HEALTH, Melbourne, Victoria (AU)

(21) Appl. No.: 14/122,946

(22) PCT Filed: May 30, 2012
(86) PCT No.: PCT/AU2012/000599

§ 371 Date: Nov. 27, 2013

(30) Foreign Application Priority Data
Jun. 1, 2011 (AU) 2011902156

(51) Int. Cl. A61B 17/68

(52) U.S. Cl. CPC A61B 17/683
USPC 606/323; 606/328

(57) ABSTRACT

A bone splint manufactured in a polylactide copolymer, the splint comprising an elongate bridging member adapted to be secured to and across fractured bone ends and means to reduce the effective length of the bridging member so that, in use, the splint holds the bone ends together.

### INTRODUCTION

This invention relates to a bone splint particularly for use in open surgical fixation of fractured ribs.

### BACKGROUND OF THE INVENTION

Rib fractures are a painful and potentially disabling injury. When multiple ribs are fractured in more than one place, a flail segment of the chest wall can occur. This injury can lead to respiratory failure requiring mechanical ventilation. The mainstay of management in these patients has traditionally been analgesia and positive pressure ventilation to splint the chest wall and allow healing of the ribs to begin. However, this management option leads to prolonged intensive care unit stay with increasing complication rates as patients remain ventilated for prolonged periods. In particular, patients are at risk of pneumonia and sepsis and will often require tracheostomy formation to aid with bronchial suctioning and weaning. Long term disabilities which have been reported in these patients include ongoing pain syndromes, inability to return to work, particularly manual type labour and cosmetic chest wall deformities.

Flail chest is a potentially life threatening injury whereby ribs which are broken in more than one place are no longer ‘fixed’ in the chest wall and become free floating. Thus during the respiratory cycle, the negative intrathoracic pressure generated during inspiration causes that segment of the chest wall to be sucked inwards rather than expanding the chest and sucking air into the lungs. This leads to paradoxical chest wall motion and impairs the mechanics of breathing which can lead to respiratory failure.

Although flail chest is the most severe manifestation of fractured ribs less severe forms of fractured ribs can still lead to severe pain and disability.

There have been proposals for operative fixation of rib fractures. Such methods include the use of anterior plates with wire cerclage, anterior plating with bicortical screws, judet struts, U-plates and absorbable plates.

However, the use of any product for rib fixation is problematic because of the repetitive movement and load bearing of the bones being fixed. It is not possible to immobilise the affected area as would be routine management in most other bone fractures. Although the ribs do not carry a heavy load, they are affected by torque in multiple directions due to the layers of intercostal muscles inserting onto the ribs. The forces impacting on the ribs are therefore constant and in multiple directions. Any rib fixation strategy needs to take these factors into account.

Most published studies of operative fixation use metal implants. Kirschner wires are prone to migration and provide no torsional stability. Pins and encircling wires have also not given reliable results in this application. Currently available metal plates are designed for other long bones and do not follow the complex curvatures of the ribs. Other drawbacks of metal plates are the need to return to theatre for removal if any complications such as infection or migration occur. Other post operative issues that can mandate removal are pain on palpation of the area particularly in thin patients, and thermal sensitivity. Further, the presence of metallic implants contradicts any future magnetic resonance imaging for the patient.

More recently, it has been suggested to use polylactide copolymers as prostheses to avoid many of the potential complications caused by metal implants.

It is these issues that have brought about the present invention.

### SUMMARY OF THE INVENTION

According to one aspect of the present invention there is provided a bone splint manufactured in a polylactide copolymer, the splint comprising an elongate bridging member adapted to be secured to and across fractured bone ends and means to reduce the effective length of the bridging member so that, in use, the splint holds the bone ends together.

Preferably, the members are located within the medullary canals of the bone ends by cement.

The means to control relative displacement may comprise serrations on the outside of the first member arranged to engage serrations on the inside of the second member, the interengagement of the serrations allowing relative displacement in one direction.

In another embodiment the means to control the relative displacement comprises a threaded interrelationship between the outside of the first member and the inside of the second member.

The first and second members are preferably hollow tubes.

The cross section of the tubes may be circular or shaped to correspond to the cross-section of the medullary canals.

In use, the splint is heated to be malleable when inserted into the bone ends.

According to a further aspect of the present invention there is provided a method of fixing fractured ribs using a splint comprising an elongate bridging member, the member being manufactured in a polylactide copolymer, the method comprising the steps of:
(a) heating the splint until malleable;
(b) securing the ends of the bridging member to end regions of adjacent bone pieces of the fractured ribs; and
(c) reducing the length of the bridging member to bring the ends together.

According to a still further aspect of the present invention there is provided a method of fixing fractured ribs using a splint comprising a first hollow member in telescopic engagement within a second hollow member, the members being manufactured in a polylactide copolymer, the method comprising the steps of:
(a) heating the splint until malleable;
(b) inserting the second member into the end of a first bone piece of the fractured rib;
(c) displacing the first member out of the second member and locating the projecting end of the first member into the bone end of a second bone piece;
(d) injecting cement into the medullary canals of the bone pieces; and (e) holding the bone ends in abutting contact until the cement sets.

Preferably the cement is injected through a hole in the wall of the first and second members until cement flows past the ends of the members to contact the bone of the medullary canals.

### DESCRIPTION OF THE DRAWINGS

Embodiments of the present invention will now be described by way of example only with reference to the accompanying drawings in which:

FIG. 1 is a perspective side view of a splint,

FIG. 2 is an end view of the splint,

FIG. 3 is a perspective view illustrating the overlapping end of inner and outer members of the splint,

FIG. 4 is an exploded side view illustrating the inner and outer members,

FIG. 5 is a side elevational view showing two broken rib pieces secured together with the splint intramedullary located,

FIG. 6 is a side elevational view of a splint in accordance with a second embodiment joining two bone ends together,

FIG. 7 is a perspective view of a C-shaped connector forming part of the splint,

FIG. 8 is a perspective view of a lateral connector for use with the splint,

FIG. 9 is a cross sectional view showing a first form of thumb tack joining the splint to bone,

FIG. 10 is a cross sectional view of the second form of thumb tack joining the splint to bone,

FIG. 11 is a sectioned perspective view showing use of the splint to join bones together,

FIG. 12 is a exploded perspective view illustrating the means of interfitting the components of the splint,

FIGS. 13 to 15 are exploded perspective views illustrating installation of a splint in accordance with another embodiment,

FIG. 16 is a cross sectional view of a plug forming part of the splint,

FIG. 17 is a cross sectional view of the plug and peg secured to a bone end, and

FIGS. 18, 19 and 20 are exploded perspective views showing installation of the splint in accordance with a further embodiment.

### DESCRIPTION OF THE PREFERRED EMBODIMENTS

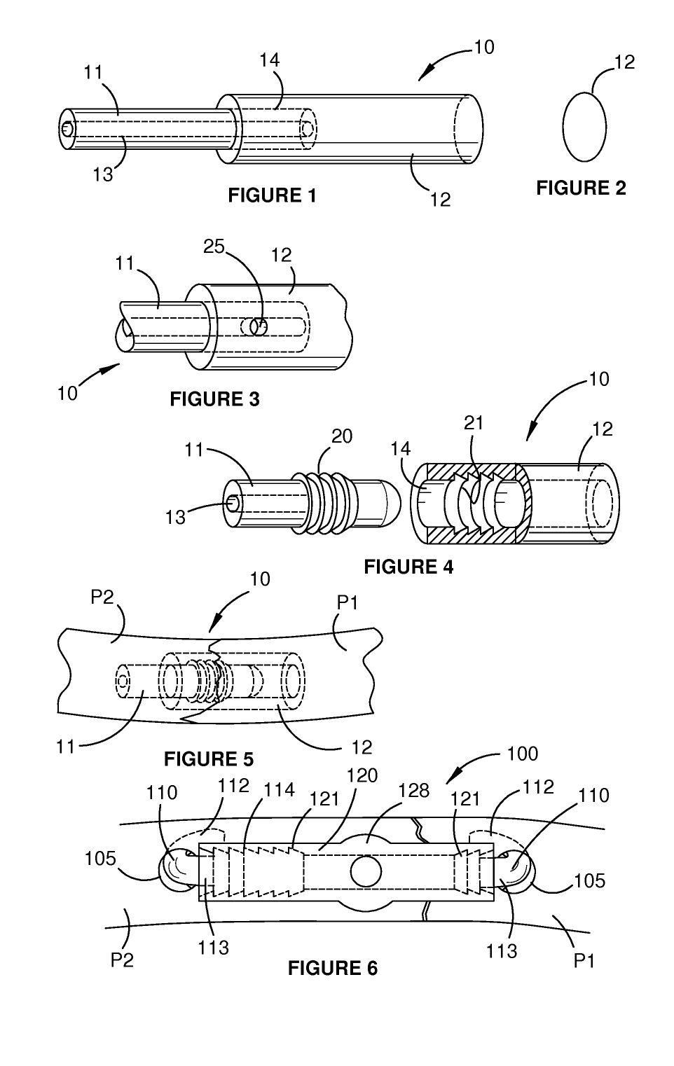

The bone splint 10 of a first embodiment is illustrated in FIGS. 1 to 5 of the attached drawings and comprises two hollow tubes 11, 12 that are extruded in a polylactide copolymer. Polylactide copolymers are, over time, absorbed by the body and are biodegradable. Polylactide copolymers become malleable when heated and then become more rigid as they cool. These copolymers maintain at least 40% of their strength for three months after insertion.

The tubes 11, 12 are hollow and comprise a first tube member 11 that is in a telescopic fit within a second tube member 12. Each member 11, 12 is 30 mm in length with an elongate throughway 13, 14 on the interior. The inter relationship between the members 11, 12 allows the tubes to be displaceable from a first withdrawn position in which the inner member 11 is wholly within the outer member 12 to an extended position shown in FIG. 1 in which the inner member extends out of the outer member 12 for 20 mm providing a splint 10 having a total length of 50 mm with a 10 mm overlap.

The cross section of rib bones are quite complex varying from the anterior to posterior ends of the rib and the inner and outer sides. In some cases the cross section of the rib bone is elliptical and in other cases it becomes virtually circular or even triangular. The cross section of the medullary canal mirrors the cross section of the exterior of the rib.

In this embodiment the splint 10 is to locate within the medullary canal of the bone pieces. The cross section of the splint is designed to mirror the cross section of the canal. This variation in cross section can be accommodated by the malleable nature of the heated polylactide copolymer or alternatively splints can be manufactured in a variety of cross sections to accommodate the varying cross sections of the rib canals. Alternatively, the splint 10 can have a simple circular cross section. This arrangement can be used when the elliptical correction proved to be a difficult fit within the medullary canals.

As shown in FIG. 2 the outer member 12 has an elliptical cross section, the longer side measuring 8.6 mm and the shorter side measuring 5.4 mm. The throughway along the length of the outer member 12 is similarly shaped with a longer dimension of 5.8 mm and a smaller dimension of 3.6 mm. The wall thickness of the outer member 12 is approximately 1.4 mm uniform around the circumference.

The inner member 11 has its outer cross section mirroring the inner cross section of the throughway 14 of the outer member 12. The longer dimension is 5.6 mm with the shorter dimension being 3.4 mm which allows the inner member 11 to be a sliding fit within the outer member 12. The inner member 11 has a central elongate throughway 13 of circular cross section with a diameter of 1.6 mm. Due to the elliptical cross section of the inner member the wall thickness varies from a minimum of 0.9 mm to a maximum of 2 mm.

A feature of the splint 10 described herein is the telescopic nature of the two members 11, 12 shown in FIG. 1. The inter relationship between the outer surface of the inner member 11 and the inner surface of the outer member 12 is such that the inner member is free to slide outwardly from a retracted position to the fully extended position, but is not free to return to the fully withdrawn position. The inter relationship is also designed to ensure that the inner member 11 cannot separate from the outer member 12.

There are a number of ways of effecting this relationship. In the embodiment shown in FIG. 4 the outer wall of the inner member 11 is serrated and these serrations 20 mirror with serrations 21 on the inner wall of the outer member 12. The inclination of the serrations 20, 21 is such that they facilitate a ratchet action whereby outward displacement is possible but inward or return displacement is prevented. The relationship at the end of the inner wall of the outer member 12 and the outer wall of the inner member 11 is such that the inner member 11 cannot be separated from the outer member 12. In another embodiment (not shown) the cross section of the throughway is the outer member 12 and the cross section of the inner member 11 are circular and there is a threaded relationship between the inner and outer members 11, 12 whereby rotation of one member relative to the other causes the inner member to move from the fully withdrawn position within the outer member 12 to the fully extended position where it projects out of the outer member 12.

It is understood that in designing the serrations 21, 20 on the inner wall of the outer member 12 and outer wall of the inner member 11, the serrations may be positioned on the poles of the oval cross section and not around the whole circumference. Alternatively the serrations may be placed around the circumference. Where serrations are used on circular members a detent may be required to prevent relative rotation of the members 11 and 12.

As shown in FIG. 5 to use the splint described above, the larger or second member 12 is first inserted into the end of the larger bone piece P1, that is, into the medullary canal. This step is carried out with the first member 11 withdrawn within the second member 12. Once the second member 12 is located in the larger bone piece the first inner member 11 is withdrawn from the outer member 12 to the fully extended position where it is slid into the medullary canal of the end of the adjacent bone piece P2.

As shown in FIG. 3 a preformed 1.6 mm hole 25 is drilled through the wall of the outer and inner members 11, 12 to communicate with the throughway 13 of the inner member 11. When the holes 25 in the outer and inner members line up the inner member is in the fully extended position. The bone ends are kept distracted and cement is then injected through the hole 25 and into the throughways of both the inner and outer members 11, 12. Sufficient cement is injected until the cement flows out of the throughways and into the medullary canals of both bone ends. When sufficient cement has been injected to ensure a significant blob at the end of each member 11, 12 the surgeon pushes the bone ends into abutting contact and hold the bone ends in place until the cement sets, which usually takes about one minute. Once the cement has set the surgeon can then release the bone ends which are then held in abutting contact by the splint 10 and its association with the cement, see FIG. 5.

Instead of preforming the holes 25 the surgeon can insert the splint 10 and then drill a hole through the larger bone piece adjacent the fractured end and through the walls of the inner and outer members 11, 12. The cement can then be injected into the splint via the drilled hole in the bone piece.

There are a number of different tools that could be used to displace the inner member 11 from the outer member 12. On the assumption that the inner member 11 is never totally within the outer member 12 a gripping tool can engage the projecting end to pull the projecting end out of the outer member 12 to the extended position. The force to pull the inner member relative to the outer member would have to resist the interengagement of the serrations. However, the malleable nature of the splint 10 when first inserted should render this displacement fairly easy.

In the embodiment where screw threads replace the serrations a suitable tool will be used to rotate the inner member 11 relative to the outer member 12 thereby causing it to project outwardly to the extended position. Another possible means of displacing the inner member relative to the outer member would be to use the cement to force the inner member out of the outer member. It is understood that in this circumstance there would have to be an abutment within the throughway of the inner member against which the cement could bear to force the inner member to move out of the outer member. It is also envisaged that the inner member could be of solid construction in which case the cement would force the inner member out by engaging the end wall of the inner member. In this embodiment it is understood that a separate source of cement would have to placed in the medullary canal of the smaller bone piece.

Another option is for the surgeon to scrape out some of the medullary cancellous bone from the ends of the fractured bone pieces and then inject a blob of cement directly into the bone ends. The splint is then inserted, placed in the extended position, and the bones held in abutting contact until the cement sets.

It is understood that the bone cement would be a conventional bone cement used in the orthopaedic industry. The bone cement would have low viscosity and designed to be fast setting.

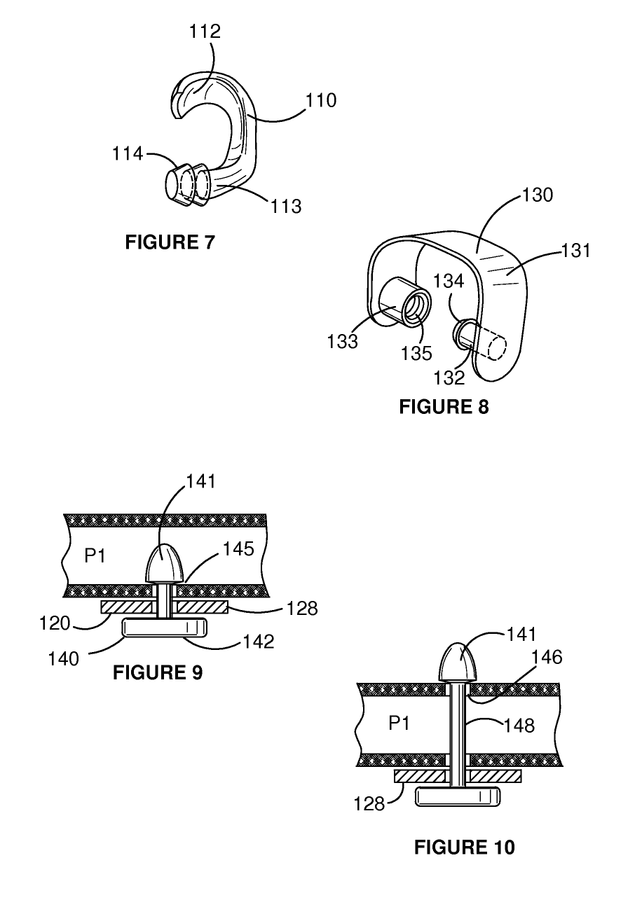

In another embodiment shown in FIGS. 6 to 12, a bone splint 100 comprises a pair of C-shaped connectors 110, that are located in a hole 105 in the bone ends drilled transversely of the bone. Each C-shaped connector has a curved arm 112 with a projecting end 113 with a serrated outer surface 114. A locking strut 120 in the form of a hollow cylinder with a serrated inner surface 121 can be pushed onto the ends 113 of the C-shaped connectors 110. The interrelationship of the serrated surfaces allows inward movement of the ends 113 of the C-shaped clips 110 into the locking strut 120 but prevents outward movement.

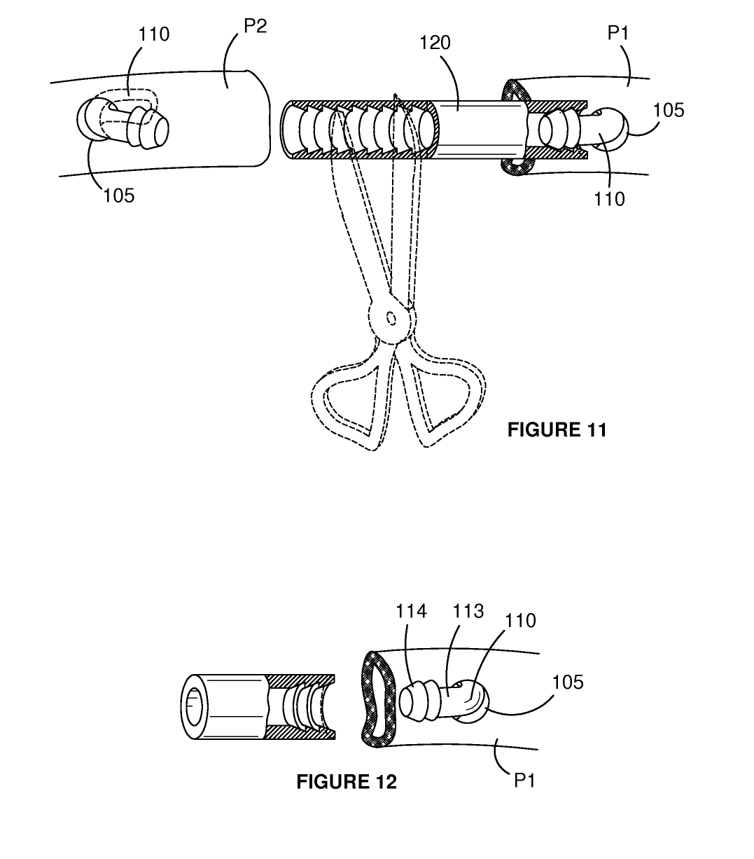

In use, the hole 105 is drilled away from the fractured end of the bone pieces P1, P2 transversely across the bone and the C-shaped connector 110 is located in the hole 105 by placing the arm 112 through the hole and manoeuvring the arm 112 through the hole 105 to engage one side of the bone with the serrated end 113 being located on the other side of the bone as shown in FIG. 6. A similar exercise is repeated for the other bone end and then the locating strut 120 is cut to length (FIG. 11). The bone ends P1, P2 are pushed together and the C-shaped connectors 110 are forced into the ends of the locating strut 120 thereby holding the bone ends in abutting contact. It is understood that the C-shaped connectors 110 and locating strut 120 are manufactured from polylactide copolymers.

To further reinforce the bone splint 100 it is understood that additional holes can be drilled through the locating strut 120 transversely of the bone ends to accommodate a locking overplate 130 (FIG. 8) comprising a U-shaped bracket 131 with internal male and female projections 132, 133. The over plate 130 is positioned over the bone and the sides of the plate are pushed inwardly so that the male projection 132 is located firmly within the female projection 133. The male projection 132 has an upstanding rib 134 that clips into a groove 135 in the female projection 133.

As shown in FIG. 9 another option includes the use of a plastics thumb tack 140 with an enlarged tapered head 141 at one end and a location flange 142 at the other. The tack 140 is forced through the locking strut 120 and into the centre of the bone through an aperture 145 drilled on one side of the bone. In another alternative shown in FIG. 10 the thumb tack 145 is longer so that the head 141 of the thumb tack extends across the bone through an aperture 146 drilled transverse to the bone to locate outside the opposite side of the bone. In this embodiment it is understood that the locking strut 120 will taper to a solid flange 128 to enable location of the thumb tacks 140. The flange 128 is shown with particular reference to FIG. 6.

It is understood that the use of thumb tacks 140 or a locking over plate 130 can be into one bone end P1 or both bone ends P1, P2 and it is further understood that in an extreme situation two location points can be positioned for the over plate 130 or thumb tacks 140 in both bone ends. The thumb tacks 140 or locking over plate 130 are only positioned in the bone ends after the bone ends have been brought into abutting contact and the locking strut 120 has been adjusted to the final position.

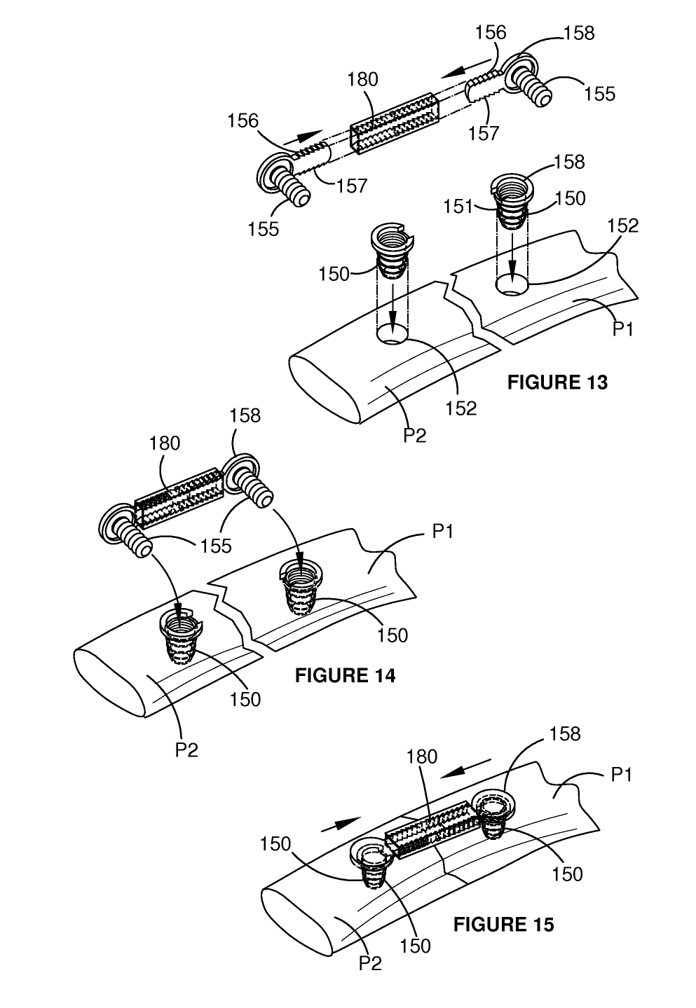

In a still further embodiment shown in FIGS. 13 to 17 the C-shaped connectors 110 are replaced by a hollow conical pin 150 with a serrated outer surface 151 which is located through a transverse hole 152 from one side of the bone P1, P2. A enlarged peg 155 can then be driven into the conical plug 150 to expand the serrated outer surface 151 against the wall of the hole 152 in the bone to improve the grip on the bone.

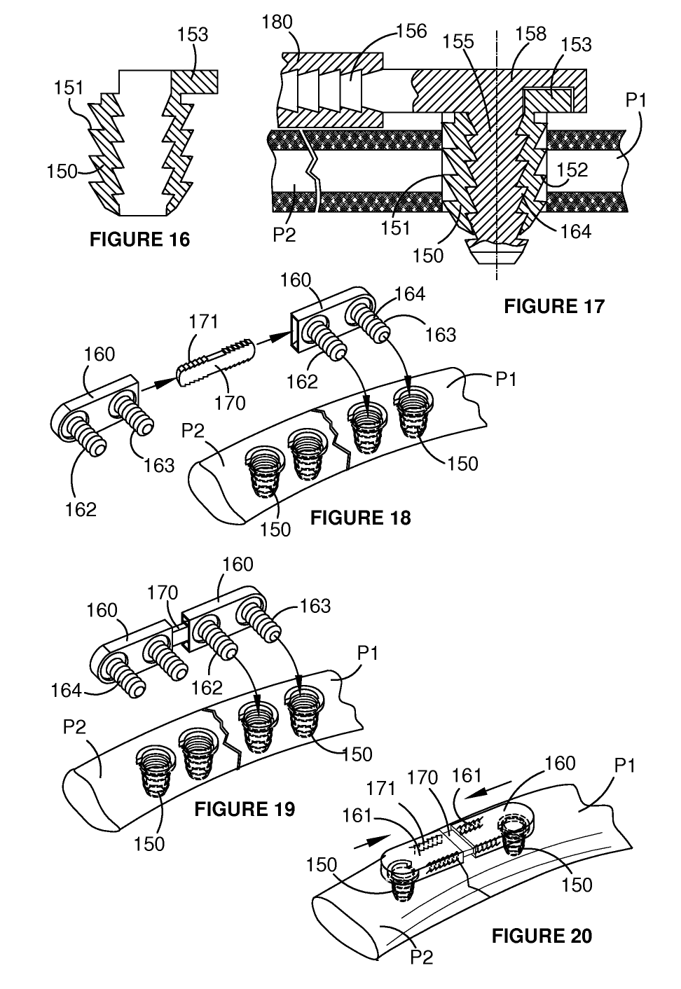

As shown in FIGS. 16 and 17 the plug 150 has a C-shaped upper flange 153. The C-shaped flange 153 accommodates the head 158 of an arm 156 with serration 157 on its exterior. The head 158 fits into the top of the plug 150. The underside of the head 158 is integrally formed with the peg 155.

In FIGS. 13 to 17 the arms 156 which are rectangular are pushed into a rectangular internally serrated bridging member 180.

In an embodiment shown in FIGS. 18 to 20, an elongate hollow rectangular receptor 160 having a serrated inner surface 161 is moulded to have two downwardly projecting pegs 162, 163 with a ribbed exterior 164. The two pegs 162, 163 can be forced into the conical pin 150 that is located in the bone to locate the receptor onto the bone end as shown in FIG. 17. This assembly is completed on both bone ends P1, P2 and when a crossbar 170 with appropriate serrations 171 on its exterior can be forced into the receptors 160 to hold the bones together. The serrated edges 171 of the cross bar 170 and receptors only allows movement of the cross bar 170 into the receptors 160 and prevent outward movement thereby holding the bone ends together.

To use the splint 100 of this embodiment the bones are first pushed together and the holes 152 drilled into the bone. The length of the receptors and crossbar is then adjusted to ensure the ends of the bone pieces remain in abutting contact.

It is understood that the receptors 160 could be held to the bone end by a single peg/pin though two spaced pegs/pins is a preferred option.

As shown in FIGS. 16 and 17 the internal surface of the plug 150 and the exterior of the peg 155 are serrated to facilitate one way movement to ensure that the peg 155 cannot escape from the plug 150.

The use of a polylactide copolymer splint avoids many of the potential complications caused by metal implants. The fact that the polylactide copolymer is malleable when heated allows the splint to assume the complex profile of the rib. Copolymers of this type maintain at least 40% of their strength after 3 months at which time the fractures would have expected to be completely healed. The copolymer is completely absorbed without toxicity over between 1 and 3 years. Animal testing has shown faster and stronger healing with absorbable splints compared to traditional metal plates which may actually slow bone healing. This occurs because the metal plate protects the bone from any load but in doing so removes the stimulus for new bone growth. In contrast the absorbable splint allows gradual transfer stress loads to the bone, stimulating faster bone growth.

An absorbable splint has the advantage that the material does not have to be removed. Furthermore, if there are complications such as migration or breakage a conservative approach can be adopted that does not warrant removal of the splint.

When the splint is warmed in hot water it becomes malleable which allows the surgeon to bend the splint to match the curve of the rib at the point of breakage. The implant has sufficient strength over its first three months to allow the bone to take up the additional stress over time. The fact that the implant becomes totally absorbed over three years avoids the likelihood of stress shielding which can cause bone weakness.

The splint described above has been specifically designed for its ease of use without the need for a second operation to later remove the splint. The simple method of fixation without screws is another significant feature of the splint.

Whilst the splint is described primarily for use in the fixation of ribs it is understood that the splint can be used on other bones, especially where the bones are not subjected to high forces and stresses. It is also understood that splints of this kind could be used in the veterinary industry on animals.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1.-14. (canceled)

15. A bone splint manufactured in a polylactide copolymer, the splint comprising an elongate bridging member adapted to be secured to and across fractured bone ends the bridging member comprising a first member in telescopic relationship with a second member, the telescopic relationship allowing movement of one member relative to the other member in one direction only and push fit fasteners to secure the first and second members to the bone ends wherein the members are pushed together to reduce the effective length of the bridging member so that, in use, the splint holds the bone ends together.

16. The splint according to claim 15 wherein serrations are formed on the outside of the first member to engage serrations on the inside of the second member, engagement of the serrations allowing relative displacement in one direction.

17. The splint according to claim 16 wherein a stop prevents separation of the first and second members.

18. The splint according to claim 15 wherein there is a threaded interrelationship between the outside of the first member and the inside of the second member.

19. The splint according to claim 15 wherein the first and second members are hollow tubes.

20. The splint according to claim 15 wherein the polylactide copolymer becomes malleable when heated.

21. The splint according to claim 15 wherein each end of the elongate bridging member is secured to a bone end region by a C-shaped hook that is a push fit through an aperture transverse of the bone ends.

22. The splint according to claim 21 wherein each C-shaped hook has a serrated end adapted to fit within a serrated end of the bridging member.

23. The splint according to claim 21 wherein the bridging member has one or more holes to accommodate the push fit fastener that is located in an aperture transverse of the bone end region.

24. The splint according to claim 23 wherein the fastener comprises a headed thumb tack.

25. The splint according to claim 23 wherein the push fit fastener comprises a tapered hollow plug that locates in the aperture in the bone and a peg that is inserted into the plug to force the plug against the wall of the aperture.

26. The splint according to claim 25 wherein the peg is integrally formed with a serrated arm that fits within the bridging member.

27. The splint according to claim 25 wherein one or more downwardly projecting pegs are integrally moulded on the underside of an internally serrated receptor and an externally serrated bridging member is arranged to be inserted into the end of the receptor.

28. The splint according to claim 25 wherein the peg exterior and plug interior are serrated to prevent escape of the peg.
